(19) United States
(12) Patent Application Publication
(10) Pub. No.: US 2007/0142254 A1
(43) Pub. Date: Jun. 21, 2007

(54) Storage system

(76) Inventors: Sandra Arce, Mine Hill, NJ (US); Silvio DiMarchi, Leonia, NJ (US); Brenda Williams-Halsey, Lake Hopatcong, NJ (US); Sheng-Fu Lee, Ramsey, NJ (US); Robert McKay, Wanaque, NJ (US); Lawrence P. Scanzillo, Upper Saddle River, NJ (US)

Correspondence Address: Darryl C. Little;Warner-Lambert Company LLC, 201 Tabor Road, Morris Plains, NJ, 07950, US

(21) Appl. No.: 11/607,228

(22) Filed: Dec. 1, 2006

Related U.S. Application Data
(60) Provisional application No. 60/742,108, filed on Dec. 2, 2005.

(51) Int. Cl. A61K 8/00

(52) U.S. Cl. 510/117

(57) ABSTRACT

The present invention relates to a storage system having (a) a container having multiple storage compartments; (b) at least one package of denture cleaning tablets provided in one of the three storage compartments; and (c) at least one additional denture cleaning resource provided in at least one other storage compartment, wherein the container is suitable for use as a tub for cleansing dentures.

### CROSS-REFERENCE TO RELATED APPLICATION

The present application claims the benefit, under 35 U.S.C. § 119(e), of the earlier filing date of U.S. provisional patent application 60/742,108, filed Dec. 2, 2005, which application is hereby incorporated herein by reference in its entirety.

### FIELD

The present invention relates to a storage system. The present invention more specifically relates to a storage system such as a storage and cleaning kit for denture cleaning that provide users with convenient, portable, stowable, and/or self-contained systems for storing denture cleaning resources.

### BACKGROUND

Denture cleaning generally is carried out either by soaking dentures in an aqueous cleansing solution and/or brushing dentures with a brush. Aqueous denture cleanser solutions are known and generally include tablets, granules, or powders that are dissolved in water to form a cleansing bath or cleansing system in water.

Denture cleansing compositions, such as effervescent tablets and powders, are known. Traditionally, these compositions have contained a variety of sulfate salts, such as bisulfates, monopersulfates, and sulfates as detergents, oxidizers and the like, and have also utilized alkali metal and alkaline earth metal halides as bleaches. Such compositions have also included perborate, carbonate and phosphate salts in varying amounts to provide effervescence and activation. Representative examples of cleansing compositions covering these various materials are set forth in U.S. Pat. Nos. 3,337,466; 3,704,227; 4,362,639; and 4,857,224. Commercial examples of denture cleaning tablets include Efferdent® denture cleanser tablets commercially available from Pfizer Consumer Healthcare of Morris Plains, N.J.

Denture cleaning tablets are generally provided to a user in boxes or containers of relatively large quantities (such as 40, 60, 120 or more tablets). Typically, 4-8 tablets are provided in an individual package or sheet. As a result, typical packaging will provide a user with 5, 10, 20 or more individual packages or sheets of denture cleaning tablets. If a user desires to use the denture cleaning tablets away from a home setting (such as while traveling), the user may bring the entire box of cleaning tablets with them. This option affords the user with containment, storage and protection of the cleaning tablets, but will likely not be practical because the user may not want to bring along unnecessary tablets and/or lose space which the box would occupy. The user could alternatively bring only one or a few individual packages with them. However, this option does not provide the user with containment, storage and protection of the cleaning tablets. The tablets may be damaged, crushed, etc., or the package may be ripped or otherwise compromised. Furthermore, a user may also require other denture cleaning resources (such as brushes for cleaning the dentures, a tub for bathing the dentures, or package openers for assisting the user to access or open the packages). Current configurations are not well suited to provide for portability, storage and/or protection of the denture cleaning tablets while also providing convenient access to and storage for other denture cleaning resources.

It would be advantageous to provide a storage system affording a portable answer to cleaning dentures as well as for storing and protecting denture cleaning tablets against damage and accidental opening. It would further be advantageous to provide a storage system allowing convenient access to other denture cleaning resources (tub, brushes, package openers, etc.) as needed. It would further be advantageous to provide a storage system that would assist in keeping the denture cleaning resources organized and protected from dirt and debris. It would further be advantageous to provide a storage system that provides a single location for all denture cleaning resources. It would be desirable to provide a system that provides any one or more of these or other advantageous features.

### SUMMARY

The present invention relates to a storage system having (a) a container having multiple storage compartments; (b) at least one package of at least one denture cleaning tablets provided in one of the storage compartments; and (c) at least one additional denture cleaning resource provided in at least one other storage compartment, wherein the container is suitable for use as a tub for denture cleansing baths.

The present invention further relates to a denture cleaning kit including (a) a container suitable for use as a tub for denture cleansing baths, the container comprising multiple compartments; (b) a denture cleaning brush provided in one compartment; and (c) at least one additional denture cleaning resource provided in at least one other storage compartment.

The present invention further relates to a denture cleaning kit including (a) a container suitable for use as a tub for denture cleansing baths, the container comprising multiple compartments; (b) at least one denture cleaning resource provided in a first of the compartments; and (c) a least one other denture cleaning resource provided in an other compartment, wherein the other compartment is configured to detachably fit within the container and is suitable for providing a denture cleaning resource.

The present invention further relates to a denture cleaning kit including (a) a container suitable for use as a tub for denture cleansing baths and having a cover, the container comprising
i.) a first compartment comprising a first article;
ii.) a second compartment comprising a second article;
iii.) a third compartment situated adjacent the first and second compartments,
   wherein the cover covers or encloses the first, second, and third compartments and wherein the cover is provided with one or more apertures in the area of the cover covering or enclosing the compartments comprising articles.

### BRIEF DESCRIPTION OF THE DRAWINGS

FIG. 1 is a front perspective view of a storage system according to an exemplary embodiment.

FIG. 2 is a front elevational view of the system shown in FIG. 1.

FIG. 3 is an exploded perspective view of the system shown in FIG. 1.

FIG. 4 is a top plan view of the system shown in FIG. 1.

FIG. 5 is a top plan view of the system shown in FIG. 1, shown in an opened state.

FIG. 6 is a left side elevational view of the system shown in FIG. 1, the right side optionally being a mirror image of the left side.

FIG. 7 is a cross-sectional view of the system shown in FIG. 1, taken along the line 7-7.

FIG. 8 is an exploded perspective view of the system shown in FIG. 1 further illustrating removable compartment 22A.

### DETAILED DESCRIPTION

Referring to the FIGURES, exemplary embodiments of a storage system are shown. According to one exemplary embodiment shown in the FIGURES and described below, the storage system is used as a denture cleaning system. The denture cleaning system provides a user with a self-contained kit to store denture cleaning resources. The system provides portability, and convenience to a user. Resources for denture cleaning are provided in a single container and may be easily transported. The system is especially well adapted as a kit to provide the user with necessary resources (such as cleaning tablets, brushes, and other accessories) for denture cleaning. The system may also be used in-home or away from home, at an office, etc.

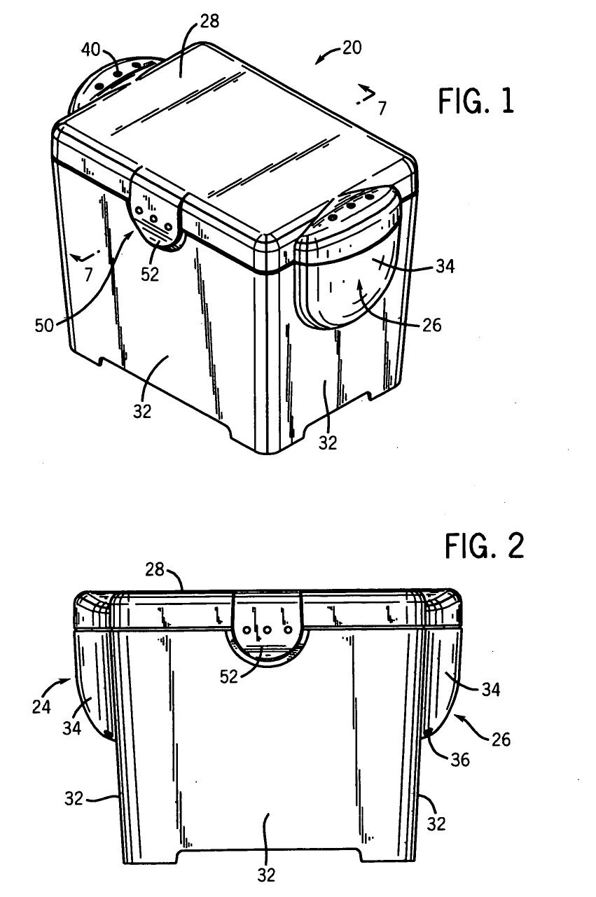

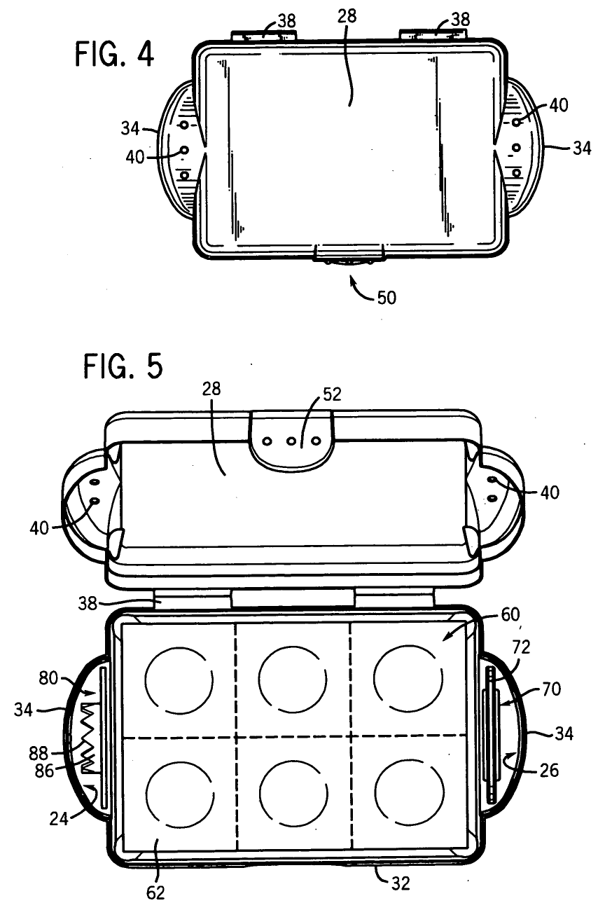

As shown in FIGS. 1-7, a storage system 10 is shown for application as a portable denture cleaning kit or for providing storage of denture cleaning resources. System 10 includes a container 20 for storage of denture cleaning resources (such as packages of denture cleaning tablets, a brush, a package opener or cutter, etc.) and where the container is useful as a tub for denture cleansing baths.

Referring to FIG. 1, container 20 provides enclosure, protection. and/or storage of the denture cleaning resources. Container 20 is shown in a closed or storage position.

Referring to FIG. 2, container 20 is shown in an open or use position. Container 20 comprises a first compartment 22 (e.g., section, bin, tub, compartment, holder or containment area), a second compartment 24 coupled to one side of first compartment 22, a third compartment 26 coupled to a second side of first compartment 22, and a cover or top portion 28.

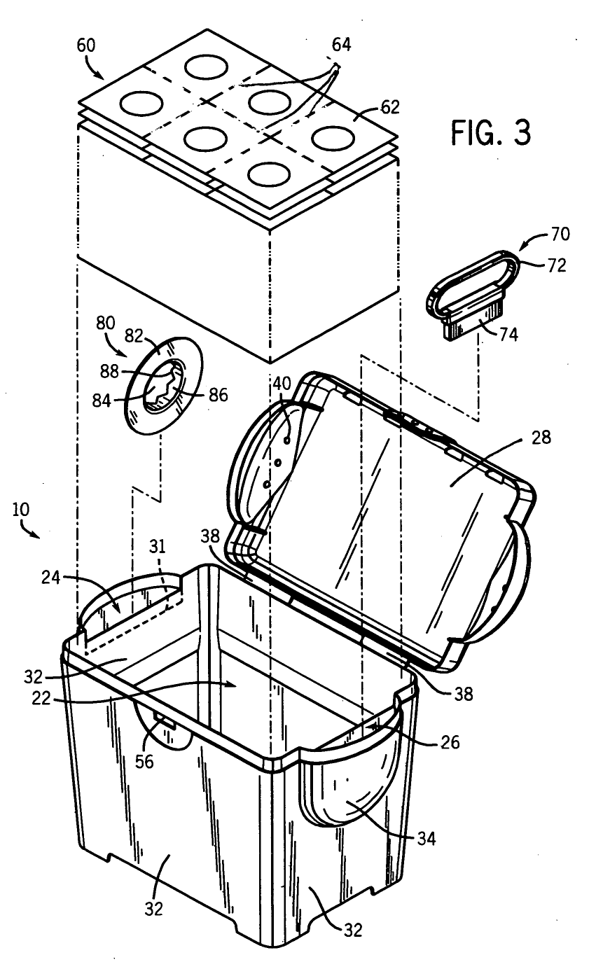

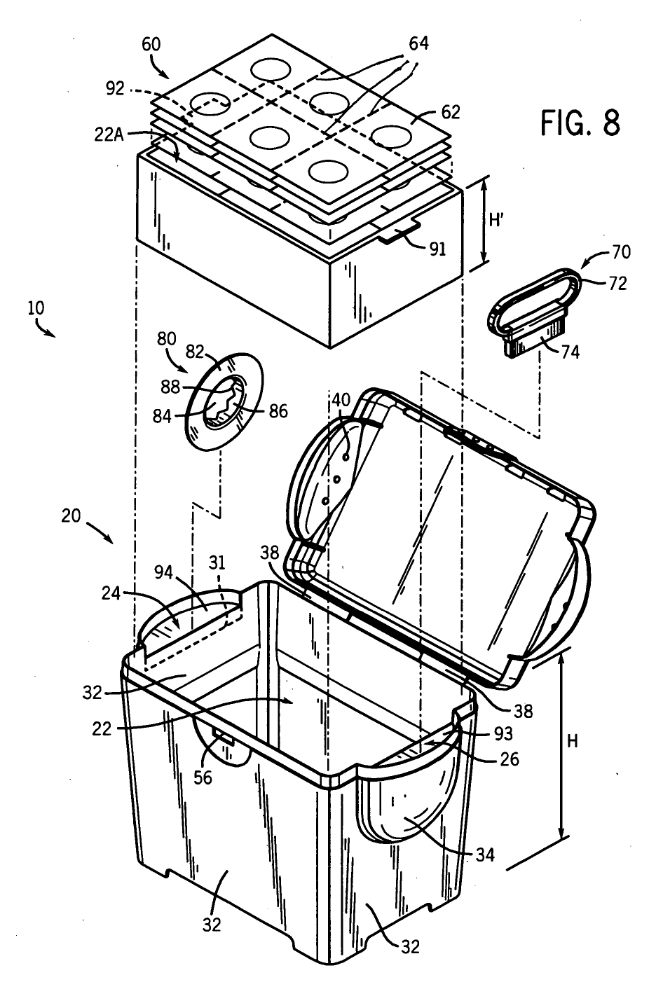

As shown in FIG. 7, first compartment 22 comprises a base or bottom 30 formed by the base or bottom of container 20, four side walls 32, and an open top through which a user may access the contents of first compartment 22. Referring back to FIG. 3, first compartment is sized to receive a stack of packages 60 of denture cleaning tablets 62 (e.g., more than one package 60 provided, in certain embodiments, in a vertical stack, on top of each other, in other embodiments stacked horizontally, etc.). Or, alternatively, container 20 is sized to receive an additional removable compartment 22A (See FIG. 8) where compartment 22A has sidewalls and a bottom and is sized to receive a stack of packages 60 of denture cleaning tablets etc. Compartment 22A would also comprise a suspending means such as that illustrated by protrusions 91 and 92 adapted to contact the top inside edges 93 and 94 of compartments 24 and 26.

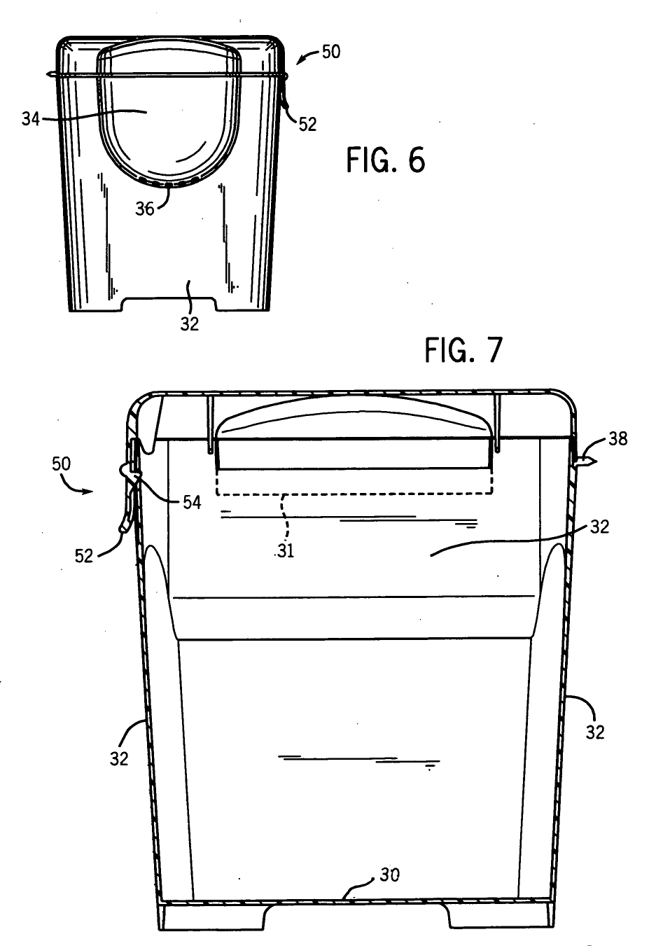

Second compartment 24 and third compartment 26 are, according to an exemplary embodiment, provided adjacent to or coupled with the first compartment 22 on opposing sides. According to an alternative embodiment, the second and third compartments may be provided on the same side of the first compartment, or in another, non-opposing arrangement. Second compartment 24 and third compartment 26 are formed by a portion of side wall 32, an outer wall 34 and an open top through which a user may access the contents of second compartment 24 and third compartment 26. To further assist a user in accessing the contents of compartments 24 and 26, a portion of side wall 32 abutting compartments 24 and 26 may be removed or otherwise not provided (e.g., not molded) to form a “cut-out” (shown as dashed line 31) and thereby providing access to compartments 24 and 26 from the side, as well as from the top. Outer wall 34 is shown as having a curved or arcuate shape in a horizontal plane (i.e., as seen in FIG. 5, outer wall 34 has a curved shape). Additionally, outer wall 34 has a curved or arcuate shape in a vertical plane (e.g., as see in FIG. 2, outer wall 34 has a curved shape). Alternatively, the outer wall may be linear in shape (thereby forming a rectangular compartment), or any other desired shape. Apertures or openings 36 may be provided in a lower portion of outer wall 34 as shown in FIGS. 2 and 6.

Referring back to FIGS. 1 and 3, cover 28 is provided to enclose or cover the open tops of compartments 22, 24, and 26. According to an exemplary embodiment, cover 28 is coupled along an edge of first compartment 22 by interface 38 as shown in FIG. 2. Interface 38 allows for movement of cover 28 relative to first compartment 22. According to an exemplary embodiment, interface 38 is a hinge, such as a living hinge that is a thin section of material coupling the cover to first compartment 22 and allowing cover 28 to be, preferably repeatedly, opened, closed, moved, rotated, etc. One or more apertures 40 are provided in cover 28, positioned such that when cover 28 is in a closed position (as shown in FIG. 1), apertures 40 are provided above, over or in the area where cover 28 covers the second compartment 24 and/or third compartment 26.

Container 20 is sized to receive a stack of denture cleaning tablet packages 60. According to an exemplary embodiment, compartment 22 has a width of about 5.75 inches, a depth of about 4 inches, and a height of about 3 inches (which is suitable to receive stack of 20 packages of denture cleaning tablets, each package having six denture cleaning tablets, the package having dimensions of about 5.5 inches in length, about 3.625 inches in width, and about 0.125 inches in depth). According to alternative embodiments, compartment 22 may vary in size and/or clearances to provide the desired operative effects (such as allowing for easier insertion, more secure fit, fitting larger or smaller packages, etc.). Also, as alluded to earlier, compartment 22 may be physically separate from container 20 as in the case of 22A in FIG. 8, but configured such that container 20 can removably receive compartment 22A.

A second compartment 24 may be provided and sized to receive and retain a brush 70 for cleaning dentures. A third compartment 26 may be provided and sized to receive and retain a package opener 80 for assisting a user open a package of denture cleaning tablets. Compartments 24 and 26 may likewise be configured for removable disposal with container 20. According to an exemplary embodiment, compartments 24 and 26 each have a maximum width of about 0.625 inches, a depth of about 2.5 inches, and a maximum height of about 2 inches.

Container 20 is secured in a locked or closed position (as shown in FIG. 1) by locking or securing mechanism 50 (such as a latch). As shown in FIGS. 3 and 7, locking mechanism 50 comprises a member 52 (such as an extension, post, tab, etc.) extending from cover 28, a protrusion 54 provided on member 52, and an aperture 56 provided in a side wall 32. In a closed position, protrusion 54 extends into aperture 56. Protrusion 54 coacts with, locks, or engages side wall 32 near an edge of aperture 56 to prevent protrusion 54 from pulling out of aperture 56, thereby keeping container 20 in a closed or locked position. To open container 20, a user will apply a force perpendicular to side wall 32 near member 52. The force will flex or otherwise move member 52 and protrusion 54 out of engagement with side wall 32. Container 20 is then free to move to an open position as shown in FIG. 3. According to an alternative embodiment, other locking mechanisms may be used to keep the container in a selectively open, closed or locked position such as hook and loop fasteners (such as Velcro® fasteners), snap closures, hooks, locks, etc.

According to an exemplary embodiment, container 20 is a single body article (i.e., unitary construction) constructed from an injection molded plastic (such as polyethylene, polypropylene, polyethylene terephthalate, etc.). According to alternative embodiments, other materials such as plastics, polymers, metals, composites, fabrics, etc. may be used. According to alternative embodiments, the container may be constructed from several separate parts attached to one another using known fasteners such as mechanical fasteners, adhesives, bonding, welding, etc.

Denture cleaning resources are stored and selectively removable from container 20. As shown in FIG. 3, denture cleaning resources include packages 60 of denture cleaning tablets 62, a denture cleaning brush 70, a package opener or cutter 80, and/or other desired resources such as swabs, wipes, strips, etc. Package 60 is a sheet-like package constructed from a foil, paper, or plastic that provides suitable boundary properties for denture cleaning tablets 62. Package 60 is shown having six individual tablets 62 provided in six individual pouches separated by score or perforation lines 64. Perforation lines 64 allow a user to tear off or separate a single tablet 62 or pouch from package 60 for use.

Package opener 80 is provided to assist a user with easily opening package 60 and gaining access to tablet 62. Package opener 80 comprises a ring-shaped body 82 having a central opening or aperture 84. Aperture 84 is sized to receive or fit a tablet 62 within aperture 84. An annular member 86 projects perpendicular to body 82. Serrations 88 (or a jagged surface, irregular surface, etc.) are provided on the end of member 86. In operation, a user will place opener 80 above tablet 62 in package 60 with the serrations 88 facing package 60. The user may then press down and/or turn opener 80 to break through, saw, or otherwise compromise package 60 and gain access to tablet 62 for use. According to an exemplary embodiment, package opener has an overall diameter of about 2 inches and an aperture diameter of about 1 inch.

Brush 70 may include handle 72 and bristles 74. According to an exemplary embodiment, handle 72 has an oval shape configured to receive one or two or more fingers of a user while operating brush 70. Bristles 74 may be any of a variety of bristles (or other abrasive elements) used to manually clean or brush dentures, teeth, etc. including nylon bristles, natural bristles, etc. According to an exemplary embodiment, brush 70 is a “finger-sized” brush, with handle 72 having dimensions of about 2 inches in width and 0.75 inches in length. Bristles 74 are provided in three rows having dimensions of about 1.25 inches in width and 0.5 inches in length.

In operation, a user will clean their dentures by opening container 20, and remove and use the desired denture cleaning resources (such as cleaning their dentures by bathing the dentures in the container tub and using the denture cleaning tablets 62 and/or the brush 70). The user will then replace the remaining denture cleaning resources and close container 20 for future use. Apertures 36 and 40 advantageously allow for the drainage of water and moisture which may be present from brush 70. Excess water retained by brush 70 may drain through apertures 36, and airflow may be promoted in compartments 24 and 26 to eliminate moisture.

According to an alternative embodiment, a storage compartment 22A may be provided for storage of additional resources desired by a user. Alternatively, the additional storage compartment may be sized and configured to receive a user's dentures or dental appliances (mouth-guards, retainers, etc.). This is easily accomplished by forming a compartment equal in size to the area provided when the height H′ of compartment 22A is less than height H of container 20.

It should be appreciated that storage system 10 affords a user with a convenient solution for storing and protecting denture cleaning tablets against damage and accidental opening and providing convenient access to other denture cleaning resources (brushes, package openers, etc.) as needed. System 10 keeps the denture cleaning resources organized and protected from dirt and debris, and providing a single location for all denture cleaning resources.

It is important to note that the construction and arrangement of the elements of the system as shown in the exemplary, particularly preferred and alternative embodiments is illustrative only. Although only a few embodiments of the present inventions have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements shown as multiple parts may be integrally formed, the operation of the locking mechanism may be reversed or otherwise varied, the length or width of the elements of the system may be varied, etc. It should be noted that the elements and/or assemblies of the system may be constructed from any of a wide variety of materials that provide sufficient strength or durability, including any of a wide variety of moldable plastic materials (such as high-impact plastic) in any of a wide variety of colors, textures and combinations, metals and composites. Accordingly, all such modifications are intended to be included within the scope of the present inventions. Other substitutions, modifications, changes and omissions may be made in the design, operating conditions and arrangement of the preferred and other exemplary embodiments without departing from the spirit of the present invention.

What is claimed is:

1. A storage system for denture cleaning articles, said storage system comprising:
a container comprising a plurality of storage compartments;
at least one package of denture cleaning tablets provided in one of said storage compartments; and
at least one additional denture cleaning resource provided in an other of said storage compartments;
wherein one of said storage compartments is suitable for use as a tub for cleansing dentures.

2. The storage system of claim 1, wherein said at least one additional denture cleaning resource comprises at least one of a brush and a package opening device.

3. The storage system of claim 2, wherein said at least one additional denture cleaning resource further comprises a brush and a package opening device.

4. The storage system of claim 1, wherein said container is at least one of semi-rigid and rigid.

5. The storage system of claim 1, further comprising a second package of denture cleaning tablets provided in one of said storage compartments.

6. The storage system of claim 1, further comprising at least one additional compartment for receiving and providing an article selected from the group consisting of dentures, dental appliances and combinations thereof.

7. The storage system of claim 6, wherein said at least one additional compartment is formed as a separate detachable compartment removably positioned in said container.

8. The storage system of claim 1, wherein said container comprises at least three storage compartments.

9. A denture cleaning kit comprising: (a) a container suitable for use as a tub for denture cleansing baths, said container comprising a plurality of compartments; (b) at least one denture cleaning resource provided in one of the said compartments; and (c) at least one other denture cleaning resource provided in another of the said compartments;
wherein the first compartment is configured to detachably fit adjacent to said other compartment within said container and is suitable for providing an article selected from the group consisting of a denture cleaning resource, dentures, a dental appliance, or a combination thereof.

10. The kit of claim 9, further comprising a cover.

11. The kit of claim 9, wherein each of said compartments further comprises:
a bottom member; and
an opposing open area through which a user may access the compartment.

12. The kit of claim 11, wherein one or more apertures are provided in said bottom member of at least one of said compartments.

13. The kit of claim 9, further comprising a locking mechanism.

14. The kit of claim 13, wherein said locking mechanism further comprises a latch.

15. A kit comprising:
a container suitable for use as a tub for denture cleansing baths, said container comprising:
i.) a first compartment containing a first article;
ii.) a second compartment containing a second article
iii.) a third compartment containing at least one package containing at least one denture cleaning tablet; and
iii.) a cover for said container, the cover covering said container, including said first, second and third compartments.

16. The kit of claim 15, wherein one or more apertures are provided in an area of said cover covering said compartments.

17. The kit of claim 15, wherein said first, second, and third compartments comprise a single unitary construction.
